(19) United States
(12) Patent Application Publication
(10) Pub. No.: US 2009/0221965 A1
(43) Pub. Date: Sep. 3, 2009

(54) INSERTION SLEEVE FOR AN INSERTION INSTRUMENT

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/394,527

(22) Filed: Feb. 27, 2009

(30) Foreign Application Priority Data
Feb. 28, 2008 (DE) 102008011688.2-44

(51) Int. Cl. A61M 25/01

(52) U.S. Cl. 604/160

(57) ABSTRACT

An insertion sleeve (1) of a medical insertion instrument, with which a catheter or balloon catheter (2) can be introduced from the outside into a blood vessel and can be moved through the blood vessel to a position to be treated and after treatment can be retracted back through the fluid-tight insertion sleeve (1). The insertion sleeve (1) is expandable over its entire effective length due to one or more breaks, for example, a continuous slit (9), in the inner wall, and furthermore it is also fluid-tight in the expanded state due to a fluid-tight, expandable casing (7) whose extent can be increased due to its elasticity and/or one or more folds (11) existing in the casing corresponding to the widening of the insertion sleeve (1).

### CROSS-REFERENCE TO RELATED APPLICATIONS

This application claims the benefit of German Application No. 10 2008 011 688.2, filed Feb. 28, 2008, which is incorporated herein by reference as if fully set forth.

### BACKGROUND

The invention relates to an insertion sleeve of a set of medical insertion instruments, with which a catheter or balloon catheter can be inserted from the outside into a blood vessel and can be moved through the blood vessel to a position in the interior of the body to be treated and after the treatment can be retracted back through the fluid-tight insertion sleeve, wherein the insertion sleeve is expandable at its distal end.

An insertion sleeve without the ability to expand and the insertion instrument to which it belongs are known from DE 34 20 455 C1. Here it is provided that the insertion sleeve is removed after the introduction of the catheter or a cardiac pacemaker electrode, while the catheter or electrode remains in the blood vessel.

Often, however, treatment catheters are inserted that must also be retracted back through the insertion sleeve when the treatment with such a treatment catheter has been completed.

Here, difficulties can arise when the treatment catheter obtains a shape that is changed relative to the insertion and that is enlarged at least in one dimension due to its use possibly, for example, at its usable end, as is possible primarily in balloon catheters. If the balloon catheter was initially inserted easily through the insertion sleeve in a folded state, but then the balloon was expanded for the treatment and then the pressurized medium were drawn off again, the balloon envelope no longer compresses exactly into the same tight form that it had for the insertion.

Thus, during retraction the balloon can cause problems at the distal end, that is, at the opening of the insertion sleeve, but also when it is pulled through the insertion sleeve, and, for example, can become hooked or remain stuck. Under some circumstances, this can even result in the need for an operation to remove the insertion instrument. It is not to be ruled out that during the retraction of such a balloon catheter, the insertion instrument or at least the insertion sleeve is ripped out simultaneously and then the resulting injury must be treated.

Indeed, from DE 91 08 043 U, an insertion instrument with an insertion sleeve is known whose distal end is similarly tapered to fit the tapering end of a dilator and can be expanded by the dilator at the distal end, but therefore does not open up the possibility of retracting the balloon of a balloon catheter back through the insertion sleeve after it has been used.

From EP 1 139 889 B1, an insertion sleeve is known that is suitable for retracting an unrolled balloon membrane through this sleeve, for which the distal section of the insertion sleeve is expandable and for this purpose has several notches at the distal end.

In this way, the drawing of a balloon for retraction in the insertion sleeve is indeed made easier, but there is the risk that the retraction of the balloon enlarged in its extent or cross section is prevented or made more difficult or even, under some circumstances, not possible due to the insertion sleeve maintaining its cross section along the rest of its further profile.

### SUMMARY

Thus there is the object of providing an insertion sleeve of the type noted above that, for constant outer dimensions, for a smallest possible insertion opening into the blood vessel, nevertheless also allows the passage of a catheter part or balloon that has become larger for its retraction.

To meet this objective, it is provided that the insertion sleeve is expandable over its entire length and is also fluid-tight in the expanded state, such that the inner sleeve wall of the insertion sleeve has, in its structure, at least one continuous break or several breaks extending in the longitudinal extent and/or breaks that are adjacent and radially continuous and the spacing of the facing edges of the break or breaks can be enlarged by expanding in the peripheral direction of the insertion sleeve and that the break or breaks is/are covered by a fluid-tight casing whose periphery can be enlarged or is expandable corresponding to the widening of the insertion sleeve.

Therefore it is possible for a balloon enlarged in volume after a treatment to also be retracted through this insertion sleeve, because this sleeve can change its cross section over its entire length in the sense of widening, but without allowing blood to escape, because it remains fluid-tight also in the expanded state.

The mentioned solution represents an especially simple and preferable possibility to make the insertion sleeve expandable not only at the distal end, but instead over its entire length, because a break that is also continuous in the radial direction in its wall permits an enlargement of the spacing of the edges of this break in the sense of widening. Simultaneously, the casing covering the breaks ensures the seal.

Indeed, the casing could cover the breaks from the inside, but it is especially preferable, also for the production, as well as for the transmission of expansion forces, when the casing is arranged on the outside of the insertion sleeve. Then it can form a good contact with this insertion sleeve and can likewise be enlarged or expanded automatically in its extent when the sleeve is widened.

An especially simple solution for an expandable insertion sleeve can provide that it has, as a break in its inner sleeve wall, at least one slit that extends over its entire length and that runs parallel to the longitudinal axis of the insertion sleeve or has a coiled configuration or a configuration with changing directions, for example, a wave-like or meander-shaped configuration, and that is open at the distal end and the fluid-tight casing covers this slit. Here, the slit extending parallel to the longitudinal axis of the insertion sleeve represents the simplest solution. According to requirements, however, the slit can also have a coiled profile, for example, like a helix, or also a wave-like or zigzag-like profile in the axial direction. In each case, the fluid-tight seal remains due to the fluid-tight casing even when this slit is widened.

It can be favorable when the casing leaves a projection of insertion sleeve at the distal end. In this way, less resistance can oppose the widening at this location.

Another solution can provide that the insertion sleeve is formed from flexible crossbars between which the breaks or intermediate spaces are arranged and that the crossbars are deformable when the insertion sleeve is widened. While typical insertion sleeves of the type according to the class must be fluid-tight overall due to their task, according to the invention the inner sleeve wall can be selected that is itself not fluid-tight, because it has the fluid-tight casing.

Accordingly, it is also possible that the insertion sleeve with its inner sleeve wall having breaks is formed from an expandable mesh and/or from expandable wire loops, as known, for example, for other medical instruments, such as expandable occlusions or stents.

The edges of the slit that can be straight or can change in direction or that is coiled can contact each other or can be spaced apart in the starting position. Here it is also possible that the spacing of the edges of the slit is constant over its length or has dimensions that change, in particular, several times. It is also possible that the boundaries of the slit do not run parallel to each other and/or in a straight line, that is, the slit can have different spacing values viewed over the length of the insertion sleeve.

The expandable casing that is especially important for the preferred embodiment of the invention can be elastic and/or have at least one fold that is folded out or unfolded when the insertion sleeve is widened. The increase in the extent of the casing can be realized in various ways, namely in that it is elastic or has at least one fold that is unfolded for the widening, wherein these two possibilities could also be combined, namely if the casing is made from an elastic material and also has at least one fold. A correspondingly large relative widening is possible if the insertion sleeve is expandable.

It can be preferable and advantageous if the casing can slide relative to the surface of the insertion sleeve. Thus, this unit made from the insertion sleeve and casing can easily expand and can have the result that a larger peripheral region or preferably the entire peripheral region of the casing is included in this expansion process and not just the part of the casing located in the slit region must take over the expansion completely or to a considerable degree. This ability to slide can be influenced by a corresponding selection of the material or materials.

For example, the casing can be a tube made, in particular, from a biocompatible material, from silicone, or from polyurethane. This produces good properties for a fluid-tight seal, expandability, and sliding ability. Natural rubber or synthetic rubber, for example, latex, can also come into play.

For good mutual sliding properties, it can be preferable if the inner sleeve wall is made from polytetrafluoroethylene (PTFE) or is coated with this material or with silicone or with polyurethane or with a biocompatible, in particular, expandable plastic.

The casing can be drawn onto the inner sleeve wall or can be co-extruded with this inner sleeve wall. Thus, there exists in advance a certain tension in the casing, namely if it has narrow dimensions relative to the insertion sleeve and is already somewhat expanded by the drawing process. In each case, co-extrusion leads to a tight and spacing-free connection and arrangement of the casing on the insertion sleeve and thus to a corresponding connection. Here it is favorable when the co-extruded materials are somewhat different, in order to allow a mutual sliding ability at a later time.

Primarily for the combination of individual or several of the features and measures described above, an insertion instrument is produced with an insertion sleeve that yields in the peripheral direction when necessary for the passage of a treatment catheter, for example, a balloon catheter, so that a balloon catheter that has become “thicker” in its dimensions after a treatment can be retracted through this insertion sleeve, because the sleeve can automatically adapt to the changed dimensions through its expandability, wherein, however, it nevertheless remains fluid-tight, which can be achieved preferably through a fluid-tight casing.

### BRIEF DESCRIPTION OF THE DRAWINGS

Embodiments of the invention are described in greater detail below with reference to the drawing. Shown in partially schematized representation are:

FIG. 1 is a side view of an insertion sleeve according to the invention of a medical insertion instrument with a slit that runs over the entire length and that opens at the distal end and that is closed fluid-tight by an expandable casing,

FIG. 2 is a cross sectional view of the insertion sleeve in the region of an enlarged diameter according to line II-II in FIG. 1, where the casing does not reach,

FIG. 3 is a perspective view showing the distal end of the insertion sleeve with its casing before the expansion,

FIG. 4 is a view similar to FIG. 3 after an expansion, wherein the casing can expand elastically,

FIG. 5 is a view according to FIG. 3, wherein the casing is modified and its expansion is possible by a reversed fold,

FIG. 6 is a view according to FIG. 4 after the insertion sleeve and the casing have been enlarged in its extent, wherein the fold of the casing is opened up or folded out,

FIG. 7 is a side view of the distal end of the insertion sleeve during the insertion of a balloon catheter that has already partially left the insertion sleeve, but is still arranged partially in the interior of the insertion sleeve,

FIG. 8 is a view according to FIG. 7 during the retraction of the balloon catheter that has been used and that has assumed or maintained a somewhat larger extent, so that the insertion sleeve wall and its casing are expanded, wherein the slit in FIGS. 1 to 8 runs parallel to the longitudinal center axis of the insertion sleeve,

FIG. 9 is a view of the distal end of an insertion sleeve with a coiled or helical line-shaped slit and an expandable casing covering this slit,

FIG. 10 is a view of the distal end of an insertion sleeve that is formed from a mesh provided with an expandable, fluid-tight casing,

FIG. 11 is a view of a modified embodiment of the insertion sleeve made from crossbars or loops in combination with a mesh,

FIG. 12 is a view of an insertion sleeve whose wall structure corresponds to that of a stent, and

FIG. 13 is a view of the distal end of an insertion sleeve whose wall is formed from crossbars or wire loops, wherein the expandable casing is left out for the sake of better clarity—like in FIG. 12.

### DETAILED DESCRIPTION OF THE PREFERRED EMBODIMENTS

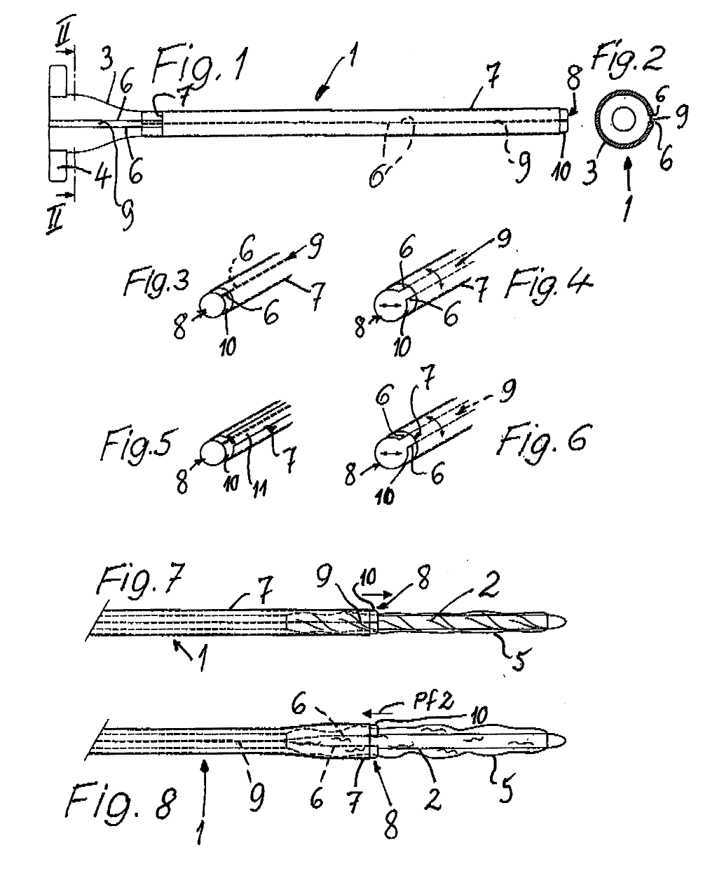

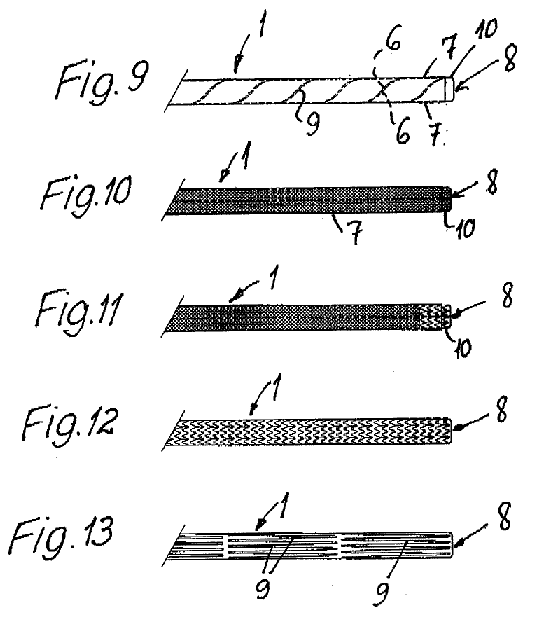

An insertion sleeve designated as a whole with 1 that is part of a medical insertion instrument is shown, with which a catheter or balloon catheter 2 (FIGS. 7 and 8) can be introduced into a blood vessel of a human body from the outside and can be moved through the blood vessel to a location in the inside of the body to be treated and after the treatment can be retracted back through the fluid-tight insertion sleeve 1. Here one sees that the largest part of the insertion sleeve has a constant, circular cross section connected to an insertion funnel 3 and a flange or handle 4 for manipulation.

In FIG. 8 it is indicated that, after treatment, a balloon catheter 2 can have a greater extent primarily in the region of its balloon 5 than for its insertion indicated schematically in FIG. 7. In the preferred embodiment the insertion sleeve is expandable over its entire length so that the balloon catheter 2 and primarily its balloon 5 now having a larger volume can be easily retracted through the insertion sleeve 2 according to the arrow PF 2, wherein, however, the insertion sleeve is also fluid-tight in the expanded state in a way still to be described. In FIG. 8 one clearly sees how the balloon 5 partially introduced into the distal end of the insertion sleeve 2 has increased, that is, widened its diameter or cross section.

Here, all of the embodiments have in common that the inner sleeve wall of the essentially cylindrical insertion sleeve 1 has, in its structure, at least one continuous break or several breaks extending in the direction of longitudinal extent of the insertion sleeve 1 and/or adjacent breaks that are continuous through the wall in the radial direction and that will be explained in even greater detail below. Here, the spacing of the edges 6 of the break or breaks can be enlarged for the expansion in the peripheral direction of the insertion sleeve 1, that is, through the expansion of the insertion sleeve 1, the edges 6 of the break or breaks are spread apart from each other.

So that here, nevertheless, no blood can escape from the blood vessel out of the insertion sleeve 1 through the break or breaks, the inner sleeve wall is enveloped or covered by a fluid-tight casing 7 whose extent can be enlarged or expanded according to the widening of the insertion sleeve 1. In FIG. 1 one sees especially well that this casing 7 extends practically over the entire length of the inner sleeve wall up to its funnel-shaped expansion 3 that is free from the casing 7. Here, in all of the embodiments one sees that the casing 7 is arranged on the outside of the insertion sleeve 1.

In the most favorable and advantageous embodiment according to FIGS. 1 to 8, the break is formed as a slit 9 that extends over the length of the region to be expanded or over the entire length of the insertion sleeve 1 and that is arranged parallel to the longitudinal axis of this sleeve and that is open at the distal end 8, and the fluid-tight casing 7 also covers this slit 9 due to the envelope of the insertion sleeve 1, wherein, at the distal end 8, a short projection 10 of the inner sleeve wall is left free from the casing 7. This simplifies the introduction of the balloon 5 after its expansion, because this projection 10 can be expanded somewhat easier without the casing 7 and in this way no blood can escape due to the introduction of the insertion sleeve into a blood vessel at this position.

FIG. 9 shows a modified embodiment at which the slit 9 is arranged coiled, that is, with a helical shape. This produces a large slit length that is also active on the periphery of the inner sleeve wall and allows a smooth and large expansion. In this case, the fluid-tight casing 7 also extends up to the projection 10.

FIG. 10 shows an insertion sleeve 1 in which the breaks in its wall are formed by an expandable mesh, while FIG. 13 shows, in several sections of the insertion sleeve 1, several slits 9 each closed at the ends as breaks that can be formed, for example, by wire loops.

FIG. 11 discloses an arrangement in which the wall of the insertion sleeve 1 is, in turn, a mesh, as known, for example, from stents that are also expandable, wherein this stent-like configuration is combined with a mesh, while FIG. 12 shows an insertion sleeve 1 that is formed similar to a stent over its entire length.

In all of the cases, the fluid-tight seal is created by the already mentioned, fluid-tight casing 7 that is elastic or has, according to FIGS. 5 and 6, at least one fold 11 that is oriented in the direction of longitudinal extent and that is folded out or unfolded for the widening according to FIG. 6 and is opened in each case, so that the widening of the casing 7 is realized by such unfolding. It is also conceivable to make the casing 7 from elastic material and also to provide a fold 11 if large expansions are to be possible relative to the diameter of the insertion sleeve 1.

The edges 6 of the slit 9 that has a straight-line configuration or a configuration changing in direction can touch each other in the starting position, as indicated in the embodiments according to FIGS. 1, 3, 5, and 7 to 9. However, it is also possible to provide these edges 6 already slightly spaced apart in the starting position, in order for primarily an elastic casing 7 to be able to better expand. In this way, the spacing of the edges 6 of the slit 9 can be constant over its length, as shown in the embodiments. This spacing, however, could also change, in particular, several times in the direction of extent of the slit 9, that is, for example, there could be regions of the slit 9 in which the edges contact each other in the starting position, while in adjacent regions the edges 6 do not contact each other, but instead have a spacing in advance.

This can also be used for good control of the expansion of the insertion sleeve 1 when the balloon 5 enlarged in its dimensions is retracted after it has been used.

So that the expansion can be performed easily and smoothly, for example, according to FIGS. 4 and 8, the casing 7 can slide relative to the surface of the inner sleeve wall, which is allowed primarily through expert material pairing. The casing 7 can be a tube made from a biocompatible material, for example, made from silicone or from polyurethane or also from polytetrafluoroethylene and the insertion sleeve itself can be made from a comparable, in particular, biocompatible material. In addition, it can be coated with an expandable plastic that keeps low or reduces the friction relative to the tube-shaped casing 7.

The casing 7 that can be seen in the embodiments is drawn onto the inner sleeve wall, but could also be co-extruded with it, in order to achieve faster production and to be able to achieve practically automatically a mutual adaptation of the dimensions of the insertion sleeve 1 and the casing 7.

The insertion sleeve 1 belongs to a medical insertion instrument, with which a catheter or a balloon catheter 2 can be introduced from the outside into a blood vessel and can be moved through the blood vessel to a position to be treated and after the treatment can be retracted back through the fluid-tight insertion sleeve 1. The inner sleeve wall is expandable at its distal end and over its entire effective length such that it has one or more breaks, for example, a continuous slit 9 and furthermore, it is also fluid-tight in the expanded state due to a fluid-tight, expandable casing 7 whose extent can be enlarged due to its elasticity and/or one or more folds 11 existing in the casing corresponding to the widening of the insertion sleeve 1.

1. Insertion sleeve (1) for a medical insertion instrument, with which a catheter or balloon catheter (2) can be introduced from outside into a blood vessel and can be moved through the blood vessel to a position in an interior of a body to be treated and after treatment can be retracted back through the insertion sleeve (1), the insertion sleeve (1) comprising an inner sleeve wall having a break or breaks defined by at least one continuous slit, several slits extending in a direction of longitudinal extent, or adjacent breaks that are continuous in a radial direction, a spacing between facing edges (6) of the break or breaks can be increased allowing the insertion sleeve (1) to widen in a peripheral direction, and the break or breaks are covered by a fluid-tight casing (7) whose extent can be increased or widened corresponding to the widening of the inner sleeve wall, such that the insertion sleeve (1) is expandable over an entire length and is also fluid-tight in the expanded state.

2. Insertion sleeve according to claim 1, wherein the casing (7) is arranged on an outside of the insertion sleeve wall.

3. Insertion sleeve according to claim 1, wherein the break or breaks comprise the at least one slit (9) that extends over the entire length and that extends parallel to a longitudinal axis, and is open on a distal end (8) and the fluid-tight casing (7) covers the slit (9).

4. Insertion sleeve according to claim 1, wherein the casing (7) does not cover a projection (10) of the inner sleeve wall at a distal end (8).

5. Insertion sleeve according to claim 1, wherein the inner sleeve is formed from flexible crossbars between which the break or breaks are arranged and the crossbars are deformable when the insertion sleeve (1) is widened.

6. Insertion sleeve according to claim 1, wherein the inner sleeve wall is formed from an expandable mesh and/or from expandable wire loops.

7. Insertion sleeve according to claim 1, wherein the break or breaks comprise the slit (9) with a configuration that is straight or that has changing directions or that is helical, and includes edges (6) that are spaced apart from each other in the starting position.

8. Insertion sleeve according to claim 7, wherein the spacing of the edges (6) of the slit (9) has dimensions that change over its length.

9. Insertion sleeve according to claim 1, wherein the expandable casing (7) is at least one of elastic or has at least one fold (11) that can be folded out or unfolded when the insertion sleeve is widened.

10. Insertion sleeve according to claim 1, wherein the casing (7) can slide relative to a surface of the inner sleeve wall.

11. Insertion sleeve according to claim 1, wherein the casing (7) is a tube made from at least one of a biocompatible material, silicone or polyurethane.

12. Insertion sleeve according to claim 11, wherein the inner sleeve wall is made from polytetrafluoroethylene (PTFE) or is coated with PTFE or with silicone or with polyurethane or with a biocompatible material.

13. Insertion sleeve according to claim 1, wherein the casing (7) is drawn onto the inner sleeve wall or is co-extruded with the inner sleeve wall.

14. Insertion sleeve according to claim 1, wherein the break or breaks comprises a slit that is helical or has changing directions in a wave-like or meander-shaped profile.

15. Insertion sleeve according to claim 1, wherein the break or breaks comprise the slit (9) with a configuration that is straight or that has changing directions or that is helical, and includes edges (6) that are in contact with each other in the starting position.
